(19) United States
(12) Patent Application Publication
(10) Pub. No.: US 2024/0358624 A1
(43) Pub. Date: Oct. 31, 2024

(54) APPLICATION TYPE BODY-CORRECTING-FILM FORMING AGENT

(71) Applicant: SHISEIDO COMPANY, LTD., Chuo-ku, Tokyo (JP)

(72) Inventors: Tomoko SATO, Tokyo (JP); Toru OKAMOTO, Tokyo (JP); Yoshiro SADAKAMI, Tokyo (JP); Akane YANAGIHARA, Tokyo (JP); Satoshi YAMAKI, Tokyo (JP); Hideo HATA, Tokyo (JP); Allison LOYND, Tokyo (JP)

(73) Assignee: SHISEIDO COMPANY, LTD., Chuo-ku, Tokyo (JP)

(21) Appl. No.: 18/682,730

(22) PCT Filed: Sep. 2, 2022
(86) PCT No.: PCT/JP2022/033151

§ 371 Date: Feb. 9, 2024

Related U.S. Application Data
(60) Provisional application No. 63/242,202, filed on Sep. 9, 2021.

(51) Int. Cl. A61K 8/895; A61Q 19/00

(52) U.S. Cl. CPC A61K 8/895; A61Q 19/007

(57) ABSTRACT

A subject matter of the present disclosure is to facilitate the permeation of a water-soluble agent into the skin.

An application type body-correcting-film forming agent of the present disclosure comprises a first agent comprising a cross-linking reactive ingredient that constitutes a body-correcting film; and a second agent comprising a catalyst for cross-linking the cross-linking reactive ingredient, wherein at least one of the first agent and the second agent comprises a water-soluble agent.

### FIELD

The present disclosure relates to an application type body-correcting-film forming agent.

### BACKGROUND

Application-type film formation agents that can be applied to the body surface to form a film that can correct wrinkles, scars, and the like are known.

Patent Literature 1 (PTL 1) discloses a composition for the in situ formation of a layer on the skin surface of a subject, comprising one or more cross-linkable polymers, and an artificial skin comprising a layer formed from the composition.

In addition, Patent Literature 2 (PTL 2) describes that water-soluble agents are agents that do not easily permeate the skin.

### CITATION LIST

### Patent Literature

[PTL 1] Japanese Translation of PCT International Application Publication No. 2019-503396
[PTL 2] Japanese Unexamined Patent Publication (Kokai) No. 2020-121938

### SUMMARY

### Technical Problem

Water-soluble agents generally permeate the skin less easily than oil-soluble agents. In particular, water-soluble agents that solidify at room temperature are even more difficult to permeate the skin because such an agent solidifies along with water evaporation after applying a composition in which the agent is dissolved in water to the skin.

Accordingly, a subject matter of the present disclosure is to facilitate the permeation of a water-soluble agent into the skin.

### Solution to Problem

### <Aspect 1>

An application type body-correcting-film forming agent comprising: a first agent comprising a cross-linking reactive ingredient that constitutes a body-correcting film; and a second agent comprising a catalyst for cross-linking the cross-linking reactive ingredient,
wherein at least one of the first agent and the second agent comprises a water-soluble agent.

### <Aspect 2>

The forming agent according to Aspect 1, wherein the water-soluble agent is a water-soluble agent that is in a solid form at room temperature.

### <Aspect 3>

The forming agent according to Aspect 2, wherein the water-soluble agent that is in a solid form at room temperature is a crystalline water-soluble agent.

### <Aspect 4>

The forming agent according to Aspect 3, wherein the crystalline water-soluble agent is at least one selected from the group consisting of 4-methoxysalicylic acid, tranexamic acid, L-ascorbic acid, 4-methoxysalicylate, tranexamate, L-ascorbate, glycylglycine, nicotinic acid amide, arbutin, L-ascorbic acid glucoside, 1-(2-hydroxyethyl)-2-imidazolidinone, and a pyrimidyl pyrazole compound represented by the following Formula 1 and a salt thereof:

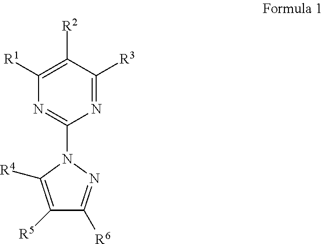

### Formula 1

where
R¹, R³, R⁴, and R⁶ are each independently an alkyl group having from 1 to 3 carbon atoms, and
R² and R⁵ are each independently a hydrogen atom or an alkyl group having from 1 to 3 carbon atoms.

### <Aspect 5>

The forming agent according to any one of Aspects 1 to 4, wherein at least one of the first agent and the second agent comprises the water-soluble agent at 0.10% by mass or more.

### <Aspect 6>

The forming agent according to any one of Aspects 1 to 5, wherein the first agent comprises at least one selected from the group consisting of a first unsaturated organopolysiloxane and a first hydride functionalized polysiloxane,
wherein when the first agent only comprises the first unsaturated organopolysiloxane out of the first unsaturated organopolysiloxane and the first hydride functionalized polysiloxane, the second agent comprises the second hydride functionalized polysiloxane, and
wherein when the first agent only comprises the first hydride functionalized polysiloxane out of the first unsaturated organopolysiloxane and the first hydride functionalized polysiloxane, the second agent comprises the second unsaturated organopolysiloxane.

### <Aspect 7>

The forming agent according to Aspect 6, wherein the first unsaturated organopolysiloxane and the second unsaturated organopolysiloxane are at least one selected from the group consisting of organopolysiloxanes containing a vinyl group, vinyl-terminated organopolysiloxanes, and organopolysiloxanes containing a vinylated branched chain.

### <Aspect 8>

The forming agent according to Aspect 7, wherein the first unsaturated organopolysiloxane and the second unsaturated organopolysiloxane are at least one selected from the group consisting of vinyl-terminated polydimethylsiloxanes, vinyl-terminated diphenylsiloxane-dimethylsiloxane copolymers, vinyl-terminated polyphenylmethylsiloxanes, vinylphenylmethyl-terminated vinylphenylsiloxane-phenylmethylsiloxane copolymers, vinyl-terminated trifluoropropylmethylsiloxane-dimethylsiloxane copolymers, vinyl-terminated diethylsiloxane-dimethylsiloxane copolymers, vinylmethylsiloxane-dimethylsiloxane copolymers, trimethylsiloxy-terminated vinylmethylsiloxane-dimethylsiloxane copolymers, silanol-terminated vinylmethylsiloxane-dimethylsiloxane copolymers, vinylmethylsiloxane homopolymers, vinyl T-structure polymers, vinyl Q-structure polymers, monovinyl-terminated polydimethylsiloxanes, vinylmethylsiloxane terpolymers, and vinylmethoxysilane homopolymers.

### <Aspect 9>

The forming agent according to any one of Aspects 6 to 8, wherein the first hydride functionalized polysiloxane and the second hydride functionalized polysiloxane are non-terminally and/or terminally hydrogenated organopolysiloxanes.

### <Aspect 10>

The forming agent according to Aspect 9, wherein the first hydride functionalized polysiloxane and the second hydride functionalized polysiloxane are at least one selected from the group consisting of hydride-terminated polydimethylsiloxanes, hydride-terminated polyphenyl-(dimethylhydrosiloxy)siloxanes, hydride-terminated methylhydrosiloxane-phenylmethylsiloxane copolymers, trimethylsiloxy-terminated methylhydrosiloxane-dimethylsiloxane copolymers, polymethylhydrosiloxanes, trimethylsiloxy-terminated polyethylhydrosiloxanes, triethylsiloxanes, methylhydrosiloxane-phenyloctylmethylsiloxane copolymers, and methylhydrosiloxane-phenyloctylmethylsiloxane terpolymers.

### <Aspect 11>

The forming agent according to any one of Aspects 1 to 10, wherein the catalyst is at least one selected from the group consisting of a platinum carbonyl cyclovinylmethylsiloxane complex, a platinum divinyltetramethyldisiloxane complex, a platinum cyclovinylmethylsiloxane complex, and a platinum octanaldehyde/octanol complex.

### <Aspect 12>

The forming agent according to any one of Aspects 1 to 11, wherein at least one of the first agent and the second agent comprises at least one selected from the group consisting of a fiber, a pigment, a dye, a thickener, a UV absorber, and a reinforcing material.

### <Aspect 13>

The forming agent according to Aspect 12, wherein the first agent comprises the reinforcing material at 0.001% by mass or more.

### <Aspect 14>

The forming agent according to any one of Aspects 1 to 13, wherein at least one of the first agent and the second agent is an emulsion composition.

### <Aspect 15>

The forming agent according to any one of Aspects 1 to 14, which is used for skin permeation of the water-soluble agent.

### <Aspect 16>

A kit comprising the first agent and the second agent in the forming agent according to any one of Aspects 1 to 15, wherein the first agent and the second agent are contained in separate containers, or contained in separate compartments of a container having two or more compartments.

### <Aspect 17>

A method for facilitating skin permeation of a water-soluble agent, comprising:
(a) preparing an application type body-correcting-film forming agent comprising: a first agent comprising a cross-linking reactive ingredient that constitutes a body-correcting film; and a second agent comprising a catalyst for cross-linking the cross-linking reactive ingredient;
(b1) applying the first agent to the skin to form a first agent layer, and then applying the second agent to the first agent layer to allow cross-linking, to form a body-correcting film;
(b2) applying the second agent to the skin to form a second agent layer, and then applying the first agent to the second agent layer to allow cross-linking, to form a body-correcting film; or
(b3) mixing the first agent and the second agent to prepare a mixture, and then applying the mixture to the skin to allow cross-linking, to form a body-correcting film,
wherein at least one of the first agent and the second agent comprises a water-soluble agent.

### Advantageous Effects of Invention

According to the present disclosure, it is possible to promote skin permeation of a water-soluble agent.

### BRIEF DESCRIPTION OF DRAWINGS

FIG. 1 is a diagram simulating the behavior of a water-soluble agent in a composition containing a water-soluble agent and moisture, and in a body-correcting film formed by a body-correcting film-forming agent containing the water-soluble agent.

### DESCRIPTION OF EMBODIMENTS

In the following, embodiments of the present disclosure will be described in detail. The present disclosure is not limited to the following embodiments, and can be implemented with a variety of variations within the scope of the main purpose of the invention.

The application type body-correcting-film forming agent of the present disclosure comprises: a first agent comprising a cross-linking reactive ingredient that constitutes a body-correcting film; and a second agent comprising a catalyst for cross-linking the cross-linking reactive ingredient, in which at least one of the first agent and the second agent comprises a water-soluble agent.

The principle of action by which the application type body-correcting-film forming agent of the present disclosure facilitates the permeation of a water-soluble agent into the skin is believed to be, but not limited to, the following.

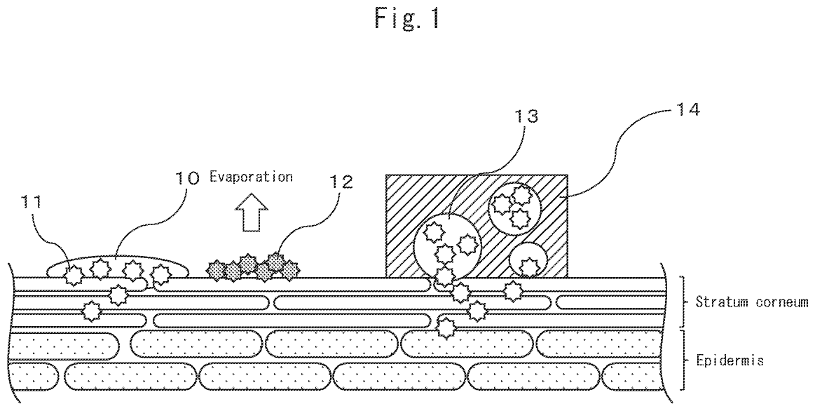

Water-soluble agents generally permeate the lipophilic skin less easily than oil-soluble agents. In addition, moisture for dissolving water-soluble agents generally evaporates more easily than oil for dissolving oil-soluble agents. Therefore, it is believed that when a composition containing a water-soluble agent and moisture is applied to the skin, as shown in FIG. 1, a part of water-soluble agent 11 dissolved in moisture 10 can slowly permeate into the skin from the composition; however, most of the water-soluble agent is dried due to evaporation of moisture in the composition to result in solid water-soluble agent 12, which does not permeate the skin but remain on the skin surface.

In an application type body-correcting-film forming agent, the components constituting a body-correcting film (sometimes simply referred to as “film”) have lipophilic properties similar to those of the skin. Therefore, in some cases, an oil-soluble agent was blended with an application type body-correcting-film forming agent that can form such a film; however, a water-soluble agent was not willingly blended with the forming agent. The present inventors have found that the skin permeability of a water-soluble agent can be improved by blending a water-soluble agent with at least one of first and second agents constituting such a forming agent to form a body-correcting film.

It is believed that when a water-soluble agent is blended with at least one of the first and second agents to form a body-correcting film, as shown in FIG. 1, the water-soluble agent is present in a state of being encapsulated by water droplet 13 in film 14. Accordingly, it is believed that unlike the composition containing a water-soluble agent and moisture, each water droplet containing the water-soluble agent is covered and sealed by the film such that the evaporation of moisture in the water droplet is reduced or suppressed, making it possible to reduce or suppress drying of the water-soluble agent, which eventually allow the water-soluble agent to permeate the skin continuously.

The film itself does not generate moisture, but while human skin is lipophilic, moisture can also be present on its surface, such as from sweat. Therefore, it is believed that water droplets in the film are guided from within the film to the skin side. Accordingly, it is believed that the water-soluble agent in the water droplets within the film is less likely to be left in the film and can permeate the skin.

The definitions of terms in the present disclosure are as follows.

“Body-correcting film” in the present disclosure means a film that is intended to exhibit a natural appearance of a skin when formed on the skin of a subject. Here, the term “natural appearance of a skin” means that the body-correcting film, when applied to the skin, exhibits a performance similar or identical to at least one selected from the external appearance, feel, and texture of the actual skin. For example, the term means that the skin subjected to the film treatment can exhibit physical properties (e.g., elasticity and firmness) of the actual (e.g., current) skin.

The term “body-correcting” in the present disclosure means that a body defect or skin defect of a subject is masked, hidden, or covered to visually and/or tactilely improve the body or skin defect, but the meaning does not include a method of operating, treating, or diagnosing a human. Here, the term “body defect” may mean, for example, an area of a subject's body that the subject perceives as a blemish or scar, or that a person skilled in the art, e.g., a dermatologist, an esthetician, or an orthopedist, considers to be a blemish or scar. The term “body defect” includes skin defects and soft tissue laxity of the body (e.g., looseness or laxity of the skin; and laxity of the breasts, buttocks, abdomen, chin, neck, and the like). The term “skin defect” includes those items of the subject's skin that the subject perceives as blemishes or scars. Examples of the skin defect include nevus flammeus or flame nevus (e.g., simple hemangioma or median flamme nevus), chloasmas, wrinkles, blemishes, acnes, moles, scars, tattoos, birthmarks, skin deformation, nevi, suntans, aging, uneven skin tones, lax skins, rough skins, hyperpigmentation, enlarged pores, telangiectasia, redness, shine, cellulite, striae gravidarum, and reduced skin elasticity.

In the present disclosure, “viscosity” refers to the measure of resistance of a fluid being deformed by either shear stress or tensile stress. For example, the viscosities of the first agent and the second agent in the application type body-correcting-film forming agent have effects on the thickness, the spreadability, and the uniformity and/or the evenness of a layer formed on a substrate. A viscosity can be reported as either a dynamic viscosity (also known as absolute viscosity; typically in the unit of Pa-s, Poise, P, or cP) or a kinematic viscosity (typically in the unit of cm²/s, Stokes, St, or cSt), and this kinematic viscosity is obtained by dividing the dynamic viscosity by the measured density of the fluid. The viscosity range of an ingredient disclosed herein is generally provided by the supplier of each ingredient as a unit of kinematic viscosity (for example, cSt), measured using a rheometer or a Cannon-Fenske tube viscometer, and the viscosity of a fluid can also be measured using, for example, a rheometer (for example, linear shear rheometer or dynamic shear rheometer) or a viscometer (also referred to as viscometer, for example, capillary viscometer or rotational viscometer).

The term “crosslinking” herein also encompasses the concept generally referred to as “curing”.

Being “water-soluble” in the present disclosure means that the octanol-water partition coefficient at 25° C. is 1.0 or less. The octanol-water partition coefficient is preferably 0 or less and more preferably −1.0 or less. Here, the octanol-water partition coefficient can be determined by adding an agent to a flask containing octanol and an aqueous buffer solution of pH 7, shaking it, and calculating the concentration of the agent in each phase using the following Formula A:

Octanol-water partition coefficient=Log₁₀(concentration of agent in octanol phase/concentration of agent in aqueous phase) Formula A.

In the present disclosure, “body surface” means the skin surface of a body.

### <<Application-Type Body-Correcting-Film Forming Agent>>

The application type body-correcting-film forming agent (simply referred to as “forming agent” in some cases) of the present disclosure comprises: a first agent comprising a cross-linking reactive ingredient that constitutes a body-correcting film; and a second agent comprising a catalyst for cross-linking the cross-linking reactive ingredient, in which at least one of the first agent and the second agent comprises a water-soluble agent. A body-correcting film obtained with a forming agent having such a configuration can improve the permeability of a water-soluble agent into the skin. In other words, the application type body-correcting-film forming agent of the present disclosure can be used for skin permeation of the water-soluble agent.

The permeability of the water-soluble agent into the skin can be evaluated by the permeability test described later using a stationary (sometimes referred to as “vertical type” or “longitudinal type”) Franz diffusion cell. When using the forming agent of the present disclosure and human skin as a membrane placed in a diffusion cell, the intramembrane concentration after 24 hours in the epidermis can be 28.0 μg/cm² or more, 30.0 μg/cm² or more, 40.0 μg/cm² or more, 50.0 μg/cm² or more, 60.0 μg/cm² or more, 70.0 μg/cm² or more, 80.0 μg/cm² or more, 90.0 μg/cm² or more, or 100.0 g/cm² or more. The upper limit of such an intramembrane concentration is not particularly limited and can be, for example, 300.0 μg/cm² or less, 250.0 μg/cm² or less, or 200.0 g/cm² or less. Here, the “intramembrane concentration” refers to the concentration of a water-soluble agent accumulated in the skin tissue (e.g., epidermis and/or dermis) during the test (intradermal concentration).

In addition, when using the forming agent of the present disclosure and human skin as a membrane placed in a diffusion cell, for example, the ratio of the intramembrane concentration after 24 hours in the skin tissue (e.g., epidermis and/or dermis) when applying the forming agent of the present disclosure containing a water-soluble agent to the skin relative to the intramembrane concentration after 24 hours in the skin tissue (e.g., epidermis and/or dermis) when applying an oil-in-water emulsion composition containing an equivalent amount of a water-soluble agent to the skin can be 1.1 or more, 1.3 or more, 1.5 or more, 2.0 or more, 3.0 or more, 4.0 or more, or 5.0 or more. The upper limit of this ratio is not particularly limited and may be, for example, 10 or less, 9.0 or less, or 8.0 or less.

In a case in which a simulated skin is used as a membrane placed in a diffusion cell, unlike human skin, it is unclear whether the simulated skin corresponds to the stratum corneum, epidermis, or dermis. Thus, the cumulative permeation amount can be used for evaluation. The term “cumulative permeation amount” used herein means the concentration of the water-soluble agent in the receptor fluid that passed through the simulated skin during the test. When using the forming agent of the present disclosure and a simulated skin as a membrane placed in a diffusion cell, the cumulative permeation amount after 24 hours can be 28.0 μg/cm² or more, 30.0 μg/cm² or more, 40.0 μg/cm² or more, 50.0 μg/cm² or more, 60.0 μg/cm² or more, 70.0 μg/cm² or more, 80.0 μg/cm² or more, 90.0 μg/cm² or more, or 100.0 μg/cm² or more. The upper limit of such a cumulative permeation amount is not particularly limited and can be, for example, 300.0 μg/cm² or less, 250.0 μg/cm² or less, or 200.0 μg/cm² or less.

In addition, when using the forming agent of the present disclosure and a simulated skin as a membrane placed in a diffusion cell, for example, the ratio of the cumulative permeation amount after 24 hours when applying the forming agent of the present disclosure containing a water-soluble agent to the simulated skin relative to the cumulative permeation amount after 24 hours when applying an oil-in-water emulsion composition containing an equivalent amount of a water-soluble agent to the simulated skin can be 1.1 or more, 1.3 or more, 1.5 or more, 2.0 or more, 3.0 or more, 4.0 or more, or 5.0 or more. The upper limit of this ratio is not particularly limited and may be, for example, 10 or less, 9.0 or less, or 8.0 or less.

In some embodiments, the application performance of an application type body-correcting-film forming agent can be evaluated in terms of viscosity using a Type B viscometer (Vismetron, manufactured by Shibaura Systems Co., Ltd.). The viscosities of the first agent and the second agent in the application type body-correcting-film forming agent of the present disclosure immediately after the preparation, measured at 25° C., 60 rpm (rotor No. 3 or No. 4), can be, for example, 100 mPa·s or more, 500 mPa·s or more, 1,000 mPa·s or more, 2,000 mPa·s or more, 5,000 mPa·s or more, 7,500 mPa·s or more, 10,000 mPa·s or more, or 15,000 mPa·s or more, and can be, for example, 1,000,000 mPa·s or less, 750,000 mPa·s or less, 500,000 mPa·s or less, 250,000 mPa·s or less, 200,000 mPa·s or less, 175,000 mPa·s or less, 150,000 mPa·s or less, 125,000 mPa·s or less, 100,000 mPa·s or less, or 80,000 mPa·s or less. Among them, from the viewpoints of smooth application performance, prevention of dripping from a skin, and the like, the first agent and the second agent in the application type body-correcting-film forming agent immediately after the preparation preferably have a viscosity of 20,000 mPa·s or less, 15,000 mPa·s or less, or 10,000 mPa·s or less, and preferably have a viscosity of 3,000 mPa·s or more, 5,000 mPa·s or more, or 7,000 mPa·s or more.

In some embodiments, from the viewpoints of smooth application performance, prevention of dripping from a skin, and the like, the viscosities of the first agent and the second agent in the application type body-correcting-film forming agent of the present disclosure 2 weeks after the preparation, measured at 25° C., 60 rpm (rotor No. 3), are preferably 50,000 mPa·s or less, 30,000 mPa·s or less, or 15,000 mPa·s or less, and are preferably 5,000 mPa·s or more, 7,000 mPa·s or more, or 10,000 mPa·s or more.

### <Water-Soluble Agent>

There is no particular restriction on the water-soluble agent, and water-soluble agents that are liquid or solid at room temperature can be used; however, in the forming agent of the present disclosure, it is advantageous to use a water-soluble agent that is solid at room temperature. Such water-soluble agents solidify when dried, further reducing their permeability into the skin, which is problematic. According to the present disclosure, the forming agent can reduce or prevent the drying of such water-soluble agents. As a result, the permeability of such agents into the skin can be improved. The water-soluble agents can be used singly or in combination of two or more kinds thereof. Here, “room temperature” is intended to mean from 0° C. to 35° C., and preferably from 20° C. to 30° C.

The water-soluble agents that are solid at room temperature can include non-crystalline (e.g., amorphous) water-soluble agents and crystalline water-soluble agents. Crystalline water-soluble agents have the problem that when they are dried and crystallized, their permeability into the skin is further reduced compared to non-crystalline water-soluble agents. According to the present disclosure, the forming agent can reduce or prevent the crystallization due to drying such crystalline water-soluble agents. As a result, the permeability of such agents into the skin can be improved. The “crystalline water-soluble agent” in the present disclosure can be intended to mean typically an ionic crystalline water-soluble agent.

Examples of such crystalline water-soluble agents can include, but are not particularly limited to, at least one selected from the group consisting of 4-methoxysalicylic acid, tranexamic acid, L-ascorbic acid, 4-methoxysalicylate, tranexamate, L-ascorbate, glycylglycine, nicotinic acid amide (niacinamide), arbutin, L-ascorbic acid glucoside, 1-(2-hydroxyethyl)-2-imidazolidinone, and a pyrimidyl pyrazole compound represented by the following Formula 1 and a salt thereof. Here, examples of the form of the salt can include, but are not particularly limited to, alkali metal salts (e.g., sodium salts, potassium salts, and lithium salts), alkaline earth metal salts (e.g., magnesium salts and calcium salts), ammonium salts, and amino acid salts, sulfates, and hydrochlorides:

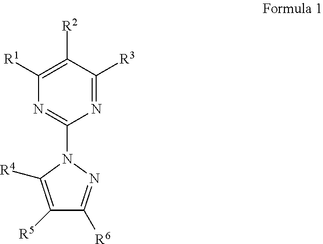

### Formula 1

where R¹, R³, R⁴, and R⁶ are each independently an alkyl group having from 1 to 3 carbon atoms, and R² and R⁵ are each independently a hydrogen atom or an alkyl group having from 1 to 3 carbon atoms.

Among the above-described water-soluble agents, 4-methoxysalicylic acid, tranexamic acid, 4-methoxysalicylate, tranexamate, and nicotinic acid amide (niacinamide) can suitably increase the permeability into the skin through the film formed by the forming agent of the present disclosure.

In addition, in some embodiments, a water-soluble agent that is allowed to permeate into the skin through the film of the present disclosure can remain in the epidermis, which is one of the stratum corneum, epidermis, and dermis which constitute the skin for a long period of time (e.g., 24 hours or more). As a result, the medicinal efficacy of such an agent on the skin (e.g., moisturizing and whitening effects) can be improved.

There is no particular restriction on the blending amount of the water-soluble agent. For example, a water-soluble agent may be blended such that it is contained in at least one of the first agent and the second agent at 0.1% by mass or more, 0.5% by mass or more, or 1.0% by mass or more. The upper limit of the blending amount is not particularly limited, and can be, for example, 10% by mass or less, 8.0% by mass or less, 5.0% by mass or less, or 3.0% by mass or less.

The water-soluble agent may be blended with at least one of the first agent and the second agent, but from the viewpoint of permeability of the water-soluble agent from the film to the skin, it is preferable that the water-soluble agent is blended with the first agent that can be the main component of the film.

### <First Agent>

The application type body-correcting-film forming agent of the present disclosure contains the first agent comprising a cross-linking reactive ingredient constituting a body-correcting film. There is no particular restriction on such a cross-linking reactive ingredient and can be, for example, at least one selected from the group consisting of a first unsaturated organopolysiloxane and a first hydride functionalized polysiloxane. It is noted that, in cases where the first agent only contains the first unsaturated organopolysiloxane out of the first unsaturated organopolysiloxane and the first hydride functionalized polysiloxane, the second agent in the forming agent of the present disclosure contains the second hydride functionalized polysiloxane described later, while in cases where the first agent only contains the first hydride functionalized polysiloxane out of the first unsaturated organopolysiloxane and the first hydride functionalized polysiloxane, the second agent contains the second unsaturated organopolysiloxane.

The dosage form of the first agent is not particularly limited. For example, the dosage form may be a single-phase system composed of an oil phase, a non-emulsifying oil-in-water or water-in-oil two-phase system, or a two-phase system configured in the form of an oil-in-water emulsion composition or a water-in-oil emulsion composition. Here, the single-phase system composed of an oil phase is typically in an anhydrous form. Here, “anhydrous” in the present disclosure is intended to mean that a composition contains no water, as well as that the water content is low, namely 10% by mass or less, 5% by mass or less, 2% by mass or less, 1% by mass or less, or 0.1% by mass or less. In addition, the non-emulsifying two-phase system may include a water-in-oil composition in which water droplets are forcibly dispersed in a dispersion medium containing oil by shaking a liquid separated into water and oil or an oil-in-water type composition in which oil droplets are forcibly dispersed in a dispersion medium containing water.

Among these dosage forms, a two-phase system configured in the form of an oil-in-water emulsion composition or a water-in-oil emulsion composition is preferable, and a water-in-oil emulsion composition is more preferable from the viewpoint of cross-linking reactivity after applying the first agent to the body surface, dispersibility of water droplets containing a water-soluble agent within the film, and skin permeability of such a water-soluble agent, and the like.

Each of these dosage forms can be prepared as appropriate by conventional methods using a cross-linking reactive ingredient and optionally known materials such as an oil, an emulsifier, and water, as described below.

Since the first agent is applied to a body surface by application or the like, the first agent preferably has a glass transition temperature that is not more than body temperature, from the viewpoint of application performance. For example, the glass transition temperature can be 37° C. or lower, 25° C. or lower, 10° C. or lower, or 0° C. or lower. The lower limit of the glass transition temperature is not particularly limited, and can be, for example, −30° C. or higher, −20° C. or higher, or −10° C. or higher. Here, “glass transition temperature” refers to a temperature at which transition from a solid state to a liquid state occurs, and can be measured, for example, using a differential scanning calorimeter (DSC) in accordance with ASTM D3418-03.

### (First Unsaturated Organopolysiloxane)

The first unsaturated organopolysiloxane is not particularly limited, and may be, for example, one or more organopolysiloxanes having at least two carbon-carbon double bonds or at least one carbon-carbon triple bond in the molecule. Preferred examples of such an unsaturated organopolysiloxane include one or more organopolysiloxanes containing at least two alkenyl functional groups on average and having a viscosity of from 10,000 to 2,000,000 cSt at 25° C. Here, “carbon-carbon double bond” and “carbon-carbon triple bond” may be referred to simply as “double bond” and “triple bond”, respectively. The first unsaturated organopolysiloxanes can be used singly or in combination of two or more kinds thereof.

Such an organopolysiloxane may contain a double bond or a triple bond in a terminal unit of the polymer, in a non-terminal monomer unit of the polymer, or in a combination thereof, particularly preferably in a non-terminal monomer unit of the polymer.

In one embodiment, double bond-containing monomer units in the organopolysiloxane may be apart from each other by, on average, 40 monomer units or more, 200 monomer units or more, 400 monomer units or more, 1,000 monomer units or more, or 2,000 monomer units or more.

In one embodiment, the amount of monomer units containing a double or a triple bond in an organopolysiloxane containing a double bond or a triple bond can be, for example, 0.01% by mass or more or 0.03% by mass or more, and can be 2% by mass or less or 0.6% by mass or less.

In one embodiment, the vinyl equivalent of organopolysiloxane containing a double bond or a triple bond can be, for example, 0.005 or more or 0.01 or more per kilogram, and can be 0.5 or less or 0.25 or less per kilogram. The approximate molar amount of double bonds or triple bonds in an organopolysiloxane can be calculated based on the average molecular weight of the organopolysiloxane. The average molecular weight or molecular mass of each ingredient disclosed herein is generally provided by the supplier of each ingredient and can be expressed in daltons (Da) or in a unit equivalent thereto, g/mol.

In one embodiment, the first unsaturated organopolysiloxane can have a viscosity of from 10,000 to 2,000,000 cSt at 25° C. The lower limit of the viscosity is preferably 20,000 cSt or more, 40,000 cSt or more, 60,000 cSt or more, 80,000 cSt or more, or 100,000 cSt or more, and more preferably 125,000 cSt or more or 150,000 cSt or more. The upper limit of the viscosity is preferably 1,000,000 cSt or less, 500,000 cSt or less, 450,000 cSt or less, 400,000 cSt or less, 350,000 cSt or less, 300,000 cSt or less, or 250,000 cSt or less, more preferably 200,000 cSt or less or 180,000 cSt or less, and still more preferably 165,000 cSt or less.

In one embodiment, the first unsaturated organopolysiloxane can have an average molecular weight of from 60,000 Da to 500,000 Da. The lower limit of such an average molecular weight is preferably 72,000 Da or more, 84,000 Da or more, 96,000 Da or more, or 100,000 Da or more, and more preferably 140,000 Da or more or 150,000 Da or more. The upper limit of the average molecular weight is preferably 200,000 Da or less, 190,000 Da or less, 180,000 Da or less, or 170,000 Da or less, more preferably 160,000 Da or less, and further preferably 155,000 Da or less.

Examples of the first unsaturated organopolysiloxane that can be used include at least one unsaturated organopolysiloxane selected from the group consisting of organopolysiloxanes containing a vinyl group, vinyl-terminated organopolysiloxanes, and organopolysiloxanes containing a vinylated branched chain.

Specific examples of the first unsaturated organopolysiloxane include vinyl-terminated polydimethylsiloxane, vinyl-terminated diphenylsiloxane-dimethylsiloxane copolymer, vinyl-terminated polyphenylmethylsiloxane, vinylphenylmethyl-terminated vinylphenylsiloxane-phenylmethylsiloxane copolymer, vinyl-terminated trifluoropropylmethylsiloxane-dimethylsiloxane copolymer, vinyl-terminated diethylsiloxane-dimethylsiloxane copolymer, vinylmethylsiloxane-dimethylsiloxane copolymer, trimethylsiloxy-terminated vinylmethylsiloxane-dimethylsiloxane copolymer, silanol-terminated vinylmethylsiloxane-dimethylsiloxane copolymer, vinylmethylsiloxane homopolymer, vinyl T-structure polymer, vinyl Q-structure polymer, monovinyl-terminated polydimethylsiloxane, vinylmethylsiloxane terpolymer, and vinylmethoxysilane homopolymer. The first unsaturated organopolysiloxane can be used singly or in combination of two or more kinds thereof. Among them, vinyl-terminated poly dimethylsiloxane is preferred, and vinyl dimethicone (divinyl dimethicone) is more preferred. In this disclosure, “terminal” is intended to mean either one or both terminals. In order to distinguish between them, they can be denoted, for example, as “one vinyl terminal” and “both vinyl terminals”.

The blending amount of the first unsaturated organopolysiloxane in the first agent may be adjusted as appropriate according to the film performance required or the like, and there is no particular restriction. For example, the blending amount of the first unsaturated organopolysiloxane with respect to the total amount of the first agent can be 5% by mass or more, 10% by mass or more, 20% by mass or more, 30% by mass or more, 35% by mass or more, or 40% by mass or more, and can be 90% by mass or less, 80% by mass or less, 70% by mass or less, 60% by mass or less, 50% by mass or less, or 45% by mass or less.

### (First Hydride Functionalized Polysiloxane)

The first hydride functionalized polysiloxane is not particularly limited, and examples thereof include a compound represented by Formula 2 below. The first hydride functionalized polysiloxane can be used singly or in combination of two or more kinds thereof.

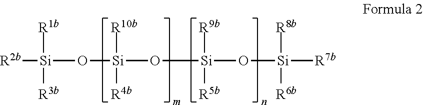

In Formula 2, R^{1b}, R^{2b}, R^{3b}, R^{4b}, R^{5b}, R^{6b}, R^{7b}, R^{8b}, R^{9b}, and R^{10b} are each independently selected from hydrogen, C₁₋₂₀ alkyl, C₂₋₂₀ alkenyl, C₅₋₁₀ aryl, hydroxyl, or C₁₋₂₀ alkoxy, and m and n are each independently an integer of 10 to 6,000, provided that at least one of R^{1b}, R^{2b}, R^{3b}, R^{4b}, R^{5b}, R^{6b}, R^{7b}, R^{8b}, R^{9b}, and R^{10b} is hydrogen.

In some embodiments, at least one of R^{1b}, R^{2b}, R^{3b}, R^{4b}, R^{5b}, R^{6b}, R^{7b}, R^{8b}, R^{9b}, and R^{10b} is hydrogen, and the rest is C₁₋₂₀ alkyl.

In some embodiments, at least two of R^{1b}, R^{2b}, R^{3b}, R^{4b}, R^{5b}, R^{6b}, R^{7b}, R^{8b}, R^{9b}, and R^{10b} are hydrogen (for example, two Si—H units per functionalized hydride polysiloxane molecule).

In other embodiments, at least three of R^{1b}, R^{2b}, R^{3b}, R^{4b}, R^{5b}, R^{6b}, R^{7b}, R^{8b}, R^{9b}, and R^{10b} are hydrogen (for example, three Si—H units per functionalized hydride polysiloxane molecule).

In some embodiments, at least two of R^{1b}, R^{2b}, R^{3b}, R^{4b}, R^{5b}, R^{6b}, R^{7b}, R^{8b}, R^{9b}, and R^{10b} is hydrogen (for example, two Si—H units per functionalized hydride polysiloxane molecule), and the rest is C₁₋₂₀ alkyl.

In other embodiments, at least three of R^{1b}, R^{2b}, R^{3b} R^{4b}, R^{5b}, R^{6b}, R^{7b}, R^{8b}, R^{9b}, and R^{10b} are hydrogen (for example, three Si—H units per functionalized hydride polysiloxane molecule), and the rest is C₁₋₂₀ alkyl.

In some embodiments, at least two of R^{4b}, R^{5b}, R^{9b}, and R^{10b} are hydrogen (for example, two Si—H units per functionalized hydride polysiloxane molecule), and the rest is C₁₋₂₀ alkyl.

In other embodiments, at least three of R^{4b}, R^{5b}, R^{9b}, and R^{10b} are hydrogen (for example, three Si—H units per functionalized hydride polysiloxane molecule), and the rest is C₁₋₂₀ alkyl.

In some embodiments, the total of m and n is an integer of 10 to 1,300, 10 to 1,100, 10 to 600, 15 to 500, 15 to 400, 20 to 300, 20 to 200, 25 to 100, 25 to 75, 30 to 50, or 40 to 45.

In some embodiments, the first hydride functionalized polysiloxane may be non-terminally and/or terminally hydrogenated organopolysiloxane, and is composed of one or more organopolysiloxanes containing at least two Si—H units in the molecule. The first hydride functionalized polysiloxane may preferably be one or more organopolysiloxanes containing at least two Si—H units on average and having a viscosity of from 2 to 100,000 cSt at 25° C.

In one embodiment, the organopolysiloxane containing an Si—H unit may contain such an Si—H unit in a terminal unit of the polymer, in a non-terminal monomer unit of the polymer, or in a combination thereof. Among them, an Si—H unit is preferably contained in a non-terminal monomer unit of the polymer. In this case, the first hydride functionalized polysiloxane may be alkyl-terminated. For example, in Formula 2, one or both of R^{2b} and R^{7b} may be C₁₋₂₀ alkyl.

In one embodiment, in Formula 2, one, two, three, four, five, or six of R^{1b}, R^{2b}, R^{3b}, R^{6b} R^{7b}, and R^{8b} may be C₁₋₂₀ alkyl.

In one embodiment, R^{1b}, R^{2b}, R^{3b}, R^{4b}, R^{5b}, R^{6b}, R^{7b}, R^{8b}, and R^{10b} each may be C₁₋₂₀ alkyl, such as C₁ alkyl (such as methyl), and R^{9b} may be hydrogen.

In one embodiment, R^{1b}, R^{2b}, R^{3b}, R^{4b}, R^{5b}, R^{6b}, R^{7b}, R^{8b}, and R^{9b} each may be C₁₋₂₀ alkyl, such as C₁ alkyl (such as methyl), and R^{10b} may be hydrogen.

In one embodiment, Si—H unit-containing monomer units in an organopolysiloxane may be, on average, 1 monomer units or more, 2 monomer units or more, 5 monomer units or more, 10 monomer units or more, 20 monomer units or more, 40 monomer units or more, 200 monomer units or more, 400 monomer units or more, 1,000 monomer units or more, or 2,000 monomer units or more apart.

In one embodiment, the amount of monomer units containing an Si—H unit in the organopolysiloxane containing an Si—H unit can be 0.003% by mass or more, 0.01% by mass or more, 0.1% by mass or more, 1% by mass or more, 3% by mass or more, 5% by mass or more, 10% by mass or more, 20% by mass or more, or 26% by mass or more, and can be 50% by mass or less, 45% by mass or less, 40% by mass or less, 35% by mass or less, 30% by mass or less, or 27% by mass or less.

In one embodiment, the Si—H content in the organopolysiloxane containing an Si—H unit can be 0.1 mmol/g or more, 0.5 mmol/g or more, 1 mmol/g or more, 2 mmol/g or more, 3 mmol/g or more, or 4 mmol/g or more, and can be 20 mmol/g or less, 10 mmol/g or less, 9 mmol/g or less, 8 mmol/g or less, 7 mmol/g or less, 6 mmol/g or less, or 5 mmol/g or less. The approximate molar amount of Si—H units in an organopolysiloxane can be calculated based on the average molecular weight of the organopolysiloxane.

In one embodiment, the first hydride functionalized polysiloxane can have a viscosity of from 2 to 500,000 cSt at 25° C. The lower limit of such a viscosity is preferably 3 cSt or more, 4 cSt or more, 5 cSt or more, 10 cSt or more, 12 cSt or more, 15 cSt or more, 20 cSt or more, 25 cSt or more, or 30 cSt or more, and more preferably 40 cSt or more. The upper limit of the viscosity is preferably 200,000 cSt or less, 100,000 cSt or less, 50,000 cSt or less, 20,000 cSt or less, 10,000 cSt or less, 5,000 cSt or less, 2,000 cSt or less, or 1,000 cSt or less, and more preferably 500 cSt or less. The viscosity of the hydride functionalized polysiloxane is particularly preferably in the range of from 45 to 100 cSt or from 45 to 50 cSt at 25° C.

In one embodiment, the hydride functionalized polysiloxane can have an average molecular weight of from 400 to 500,000 Da. The lower limit of such an average molecular weight is preferably 500 Da or more, 800 Da or more, 900 Da or more, 1,000 Da or more, 1,200 Da or more, 1,400 Da or more, 1,600 Da or more, 1,800 Da or more, 2,000 Da or more, or 2,200 Da or more, and more preferably 2,300 Da or more. The upper limit of the average molecular weight is preferably 250,000 Da or less, 140,000 Da or less, 100,000 Da or less, 72,000 Da or less, 62,700 Da or less, 60,000 Da or less, 50,000 Da or less, 49,500 Da or less, 36,000 Da or less, 28,000 Da or less, 25,000 Da or less, 20,000 Da or less, 15,000 Da or less, 10,000 Da or less, 5,000 Da or less, or 4,000 Da or less, and more preferably 2,500 Da or less.

The first hydride functionalized polysiloxane that can be employed may be, for example, but not limited to, at least one selected from the group consisting of hydride-terminated polydimethylsiloxane, hydride-terminated polyphenyl-(dimethylhydrosiloxy)siloxane, hydride-terminated methylhydrosiloxane-phenylmethylsiloxane copolymer, trimethylsiloxy-terminated methylhydrosiloxane-dimethylsiloxane copolymer, polymethylhydrosiloxane, trimethylsiloxy-terminated polyethylhydrosiloxane, triethylsiloxane, methylhydrosiloxane-phenyloctylmethylsiloxane copolymer, and methylhydrosiloxane-phenyloctylmethylsiloxane terpolymer. Among them, hydride-terminated polydimethylsiloxane is preferred, and hydrogen dimethicone is more preferred.

The blending amount of the first hydride functionalized polysiloxane in the first agent may be adjusted as appropriate according to the film performance required or the like, and there is no particular restriction. For example, the blending amount of the first hydride functionalized polysiloxane with respect to the total amount of the first agent can be 1% by mass or more, 3% by mass or more, or 5% by mass or more, and can be 75% by mass or less, 60% by mass or less, 50% by mass or less, 40% by mass or less, 30% by mass or less, 20% by mass or less, or 10% by mass or less.

### (Other Polymers)

The first agent can optionally contain polymers other than the first unsaturated organopolysiloxane and the first hydride functionalized polysiloxane. The other polymers can be used singly or in combination of two or more kinds thereof.

In one embodiment, the other polymers can have a viscosity of from 0.7 cSt to 50,000 cSt at 25° C. The lower limit of such a viscosity can be 1 cSt or more, 6 cSt or more, 10 cSt or more, 20 cSt or more, 50 cSt or more, 100 cSt or more, 200 cSt or more, 300 cSt or more, 400 cSt or more, 750 cSt or more, 1,000 cSt or more, 1,500 cSt or more, 2,000 cSt or more, 2,500 cSt or more, 3,000 cSt or more, 3,500 cSt or more, or 4000 cSt or more. The upper limit of the viscosity can be 45,000 cSt or less, 40,000 cSt or less, 35,000 cSt or less, 30,000 cSt or less, 25,000 cSt or less, 20,000 cSt or less, 15,000 cSt or less, 12,000 cSt or less, 10,000 cSt or less, 5,000 cSt or less, 4,000 cSt or less, 2,000 cSt or less, 1,500 cSt or less, or 1,000 cSt or less.

In one embodiment, the other polymers can have an average molecular weight of from 180 Da to 80,000 Da. The lower limit of such an average molecular weight can be 500 Da or more, 800 Da or more, 1,500 Da or more, 3,000 Da or more, 6,000 Da or more, 9,400 Da or more, 10,000 Da or more, 15,000 Da or more, 20,000 Da or more, 30,000 Da or more, 40,000 Da or more, 50,000 Da or more, 55,000 Da or more, 60,000 Da or more, or 62,000 Da or more. The upper limit of the average molecular weight can be 75,000 Da or less, 70,000 Da or less, 65,000 Da or less, or 63,000 Da or less.

Preferred examples of the other polymers include one or more organopolysiloxanes containing at least one alkenyl functional group on average and having a viscosity of from 0.7 to 50,000 cSt at 25° C.

Specific examples of the other polymers that can be used include at least one selected from the group consisting of vinyl-terminated polydimethylsiloxane, vinyl-terminated diphenylsiloxane-dimethylsiloxane copolymer, vinyl-terminated polyphenylmethylsiloxane, vinylphenylmethyl-terminated vinylphenylsiloxane-phenylmethylsiloxane copolymer, vinyl-terminated trifluoropropylmethylsiloxane-dimethylsiloxane copolymer, vinyl-terminated diethylsiloxane-dimethylsiloxane copolymer, vinylmethylsiloxane-dimethylsiloxane copolymer, trimethylsiloxy-terminated vinylmethylsiloxane-dimethylsiloxane copolymer, silanol-terminated vinylmethylsiloxane-dimethylsiloxane copolymer, vinyl-terminated vinyl rubber, vinylmethylsiloxane homopolymer, vinyl T-structure polymer, vinyl Q-structure polymer, an unsaturated organic polymer (such as an unsaturated fatty alcohol, an unsaturated fatty acid, an unsaturated fatty ester, an unsaturated fatty amide, an unsaturated fatty urethane, an unsaturated fatty urea, ceramide, crocetin, lecithin, and sphingosine), monovinyl-terminated poly dimethylsiloxane, vinylmethylsiloxane terpolymer, vinylmethoxysilane homopolymer, vinyl-terminated poly alkylsiloxane polymer, and vinyl-terminated polyalkoxysiloxane polymer. Among them, vinyl-terminated polydimethylsiloxane is preferred, and divinyl dimethicone and 1,3-divinyltetramethyl disiloxane are more preferred.

The blending amount of other polymers in the first agent may be adjusted as appropriate according to the film performance required or the like, and there is no particular restriction. For example, the blending amount of other polymers with respect to the total amount of the first agent can be 0.01% by mass or more, 0.1% by mass or more, 0.3% by mass or more, or 0.5% by mass or more, and can be 20% by mass or less, 15% by mass or less, or 10% by mass or less.

### <Ratios of Functional Groups in First Unsaturated Organopolysiloxane, First Hydride Functionalized Polysiloxane, and Other Polymers>

In one embodiment, the molar ratio of the Si—H functional groups derived from the first hydride functionalized polysiloxane to the alkenyl functional groups derived from the first unsaturated organopolysiloxane is preferably from 60:1 to 1:5, and more preferably from 45:1 to 15:1.

In one embodiment, the molar ratio of the Si—H functional groups derived from the first hydride functionalized polysiloxane to the alkenyl functional groups derived from other polymers is preferably from 60:1 to 1:5, and more preferably from 45:1 to 15:1.

In one embodiment, the molar ratio of the alkenyl functional groups derived from the first unsaturated organopolysiloxane to the alkenyl functional groups derived from the other polymers is preferably from 100:1 to 1:100, and more preferably from 10:1 to 1:10.

### <Second Agent>

The second agent that constitutes the application type body-correcting-film forming agent of the present disclosure contains a catalyst for cross-linking the cross-linking reactive ingredient in the first agent described above.

### (Catalyst)

The catalyst is not particularly limited, and can be, for example, any substance capable of causing, promoting, or initiating a physical and/or chemical cross-linking reaction targeting the unsaturated organopolysiloxane and the hydride functionalized polysiloxane that are cross-linking reactive ingredients constituting the body-correcting film. The catalyst optionally undergoes permanent physical and/or chemical changes during or at the end of the process.

The catalyst can include, but is not limited to, a metal catalyst capable of initiating and/or promoting cross-linking at or below body temperature, and examples thereof include a Group VIII metal catalyst such as a platinum catalyst, a rhodium catalyst, a palladium catalyst, a cobalt catalyst, a nickel catalyst, a ruthenium catalyst, an osmium catalyst, or an iridium catalyst and a Group IVA metal catalyst such as a germanium catalyst or a tin catalyst. Among them, a platinum catalyst, a rhodium catalyst, or a tin catalyst is preferred. The catalysts can be used singly or in combination of two or more kinds thereof.

Examples of a platinum catalyst include a platinum carbonyl cyclovinylmethylsiloxane complex, a platinum divinyltetramethyldisiloxane complex, a platinum cyclovinylmethylsiloxane complex, a platinum octanaldehyde/octanol complex, and another Pt(0) catalyst such as a Karstedt's catalyst, a platinum-alcohol complex, a platinum-alkoxide complex, a platinum-ether complex, a platinum-aldehyde complex, a platinum-ketone complex, a platinum-halogen complex, a platinum-sulfur complex, a platinum-nitrogen complex, a platinum-phosphorus complex, a platinum-carbon double-bond complex, a platinum-carbon triple-bond complex, a platinum-imide complex, a platinum-amide complex, a platinum-ester complex, a platinum-phosphate complex, a platinum-thiol ester complex, a platinum-isolated electron pair complex, a platinum-aromatic complex, or a platinum-p-electron complex, and a combination thereof. Among them, at least one selected from the group consisting of a platinum carbonyl cyclovinylmethylsiloxane complex, a platinum divinyltetramethyldisiloxane complex, a platinum cyclovinylmethylsiloxane complex, and a platinum octanaldehyde/octanol complex is preferred.

Examples of a rhodium catalyst include tris(dibutyl sulfide)rhodium trichloride and rhodium trichloride hydrate.

Examples of a tin catalyst include tin (II) octanoate, tin (II) neodecanoate, dibutyltin diisooctylmaleate, di-n-butylbis(2,4-pentanedionate)tin, di-n-butylbutoxychlorotin, dibutyltin dilaurate, dimethyltin dineodecanoate, dimethylhydroxy(oleate)tin, and tin (II) oleate.

Among these catalysts, a platinum catalyst is more preferred, and a platinum divinyltetramethyldisiloxane complex is particularly preferred.

The blending amount of the catalyst in the second agent may be adjusted as appropriate according to the film performance or the like required, and is not particularly limited. For example, the blending amount of the catalyst with respect to the total amount of the second agent can be 0.001% by mass or more, 0.005% by mass or more, or 0.010% by mass or more, and can be 1.0% by mass or less, 0.10% by mass or less, or 0.050% by mass or less.

### <Dosage Form of Second Agent>

The dosage form of the second agent in the present disclosure is not particularly limited. For example, the dosage form may be a single-phase system composed of an oil phase in the anhydrous form, a non-emulsifying oil-in-water or water-in-oil two-phase system, or a two-phase system configured in the form of an oil-in-water emulsion composition or a water-in-oil emulsion composition. Among these, a two-phase system configured in the form of an oil-in-water emulsion composition or a water-in-oil emulsion composition is preferable from the viewpoint of cross-linking reactivity with the first agent, dispersibility of water droplets containing a water-soluble agent within the film, and skin permeability of such a water-soluble agent, and the like.

Each of these dosage forms can be prepared as appropriate by conventional methods using a catalyst and optionally known materials such as an oil, an emulsifier, and water, as described below.

### (Oil)

Examples of the oil can include liquid oils and fats, solid oils and fats, waxes, hydrocarbon oils, silicone oils, and polar oils. The oils can be used singly or in combination of two or more kinds thereof.

Examples of a silicone oil that can be used include chain silicones such as dimethylpolysiloxane (dimethicone), methylphenylpolysiloxane, and methylhydrogen polysiloxane; and cyclic silicones such as octamethyl cyclotetrasiloxane, decamethyl cyclopentasiloxane, and dodecamethyl cyclohexasiloxane. It is also possible to use a first unsaturated organopolysiloxane and a first hydride functionalized polysiloxane, which may be used in the above-described first agent, as a silicone oil. To distinguish from the first unsaturated organopolysiloxane and the first hydride functionalized polysiloxane in the first agent, the unsaturated organopolysiloxane and hydride functionalized polysiloxane in the second agent can be referred to as the “second unsaturated organopolysiloxane” and “second hydride functionalized polysiloxane”, respectively.

There is no particular restriction on the blending amount of oil, and it can be adjusted as appropriate depending on, for example, the type of dosage form employed.

### (Emulsifier)

As the emulsifier, for example, an anionic, cationic, amphoteric, or nonionic emulsifier can be used. The emulsifiers can be used singly or in combination of two or more kinds thereof. Here, the emulsifier in the present disclosure means an agent having an emulsifying function (surface-active properties), and can also include an agent generally called surfactant. The above-described water-soluble agent does not encompass an emulsifier.

Specific examples of the emulsifier include at least one selected from the group consisting of hydrocarbon surfactants, silicone surfactants and amphiphilic powders.

Examples of the hydrocarbon surfactants include polyoxyethylene alkyl ethers, polyoxyethylene steryl ethers, polyoxyethylene fatty acid esters, polyoxyethylene polyhydric alcohol fatty acid esters, polyoxyethylene hydrogenated castor oils, polyoxyethylene sorbitan fatty acid esters, glycol fatty acid esters, glycerol fatty acid esters, sorbitan fatty acid esters, sucrose fatty acid esters, and polyglycerol fatty acid esters.

Examples of the silicone surfactants include polyether-modified silicones, and alkyl co-modified polyether-modified silicones.

The blending amount of the emulsifier is not particularly limited, and can be, for example, 0.01% by mass or more, 0.05% by mass or more, 0.1% by mass or more, or 0.2% by mass or more with respect to the total amount of the second agent from the viewpoint of emulsion stability and the like. The upper limit of the blending amount of the emulsifier is not particularly limited, and can be, for example, 5% by mass or less, 4% by mass or less, 3% by mass or less, 2% by mass or less, or 1% by mass or less.

### (Water)

Water is not particularly limited, and for example, water used in cosmetics or quasi-drugs can be used. For example, ion-exchanged water, distilled water, ultrapure water, and tap water can be used.

There is no particular restriction on the blending amount of water, and it can be adjusted as appropriate depending on, for example, the type of dosage form employed.

### (Other Polymers)

The above-described other polymers that can be blended in the first agent can be similarly blended in the second agent.

When the second unsaturated organopolysiloxane and the other polymers are used in the second agent, the functional-group ratio between these that may be employed is the same as the functional-group ratio between the first unsaturated organopolysiloxane and the other polymers.

### <Optional Ingredients>

The application type body-correcting-film forming agent of the present disclosure may contain various ingredients in the first agent and/or the second agent as appropriate as long as the effects of the present disclosure are not negatively affected.

The optional ingredients are not particularly limited, and examples thereof include a feel modifier, a pressure-sensitive adhesion modifier, a spreading promoter, a diluent, an adhesion modifier, an emulsifier (surfactant) other than the above, a solvent, a humectant, a preservative, a fiber, a pigment, a dye, an ingredient for thickening the aqueous phase or oil phase (thickener), a protective colloid, a reinforcing material (filler), a skin permeation enhancer, an optical modifier, a scattering agent, an adsorbent, a magnetic material, a gaseous transport modifier, a liquid transport modifier, a pH modifier, a sensitizing modifier, and an aesthetic modifier. Such optional ingredients can be used singly or in combination of two or more kinds thereof. Among the optional ingredients, water-soluble ingredients may be encompassed in the above-described water-soluble agent.

In addition, examples of optional ingredients can include: cosmetic agents such as a moisturizer, a UV absorber, a skin protectant, a skin calming agent, a skin whitener, a skin brightener, a skin softener, a skin smoothing agent, a skin bleaching agent, a skin exfoliator, a skin tightener, a beauty agent, a vitamin, an antioxidant, a cell signaling agent, a cell modulator, a cell interaction agent, a skin tanning agent, an anti-aging agent, an anti-wrinkle agent, a spot reducer, an α-hydroxy acid, a β-hydroxy acid, and a ceramide; therapeutic agents such as a pain reliever, an analgesic, an antipruritic agent, an anti-acne agent (such as β-hydroxy acid, salicylic acid, or benzoyl peroxide), an anti-inflammatory agent, an antihistamine, a corticosteroid, a NSAID (nonsteroidal anti-inflammatory drug), an antiseptic, an antibiotic, an antibacterial agent, an antifungal agent, an antiviral agent, an antiallergic agent, an anti-irritant agent, an insect repellent, a phototherapeutic agent, a blood coagulant, an anti-neoplastic agent, an immune system stimulant, an immune system suppressant, a coal tar, anthralin, fluocinonide, methotrexate, cyclosporine, pimecrolimus, tacrolimus, azathioprine, fluorouracil, a ceramide, a counterirritant, and a skin-cooling compound; and agents such as an antioxidant, a vitamin, a vitamin D₃ analog, a retinoid, a mineral, a mineral oil, vaseline, a fatty acid, a plant extract, a polypeptide, an antibody, a protein, a sugar, a humectant, and an emollient.

Examples of the above-described reinforcing material can include at least one selected from carbon (such as graphene), silver, mica, zinc sulfide, zinc oxide, titanium dioxide, aluminum oxide, clay (such as kaolin), chalk, talc, calcite (such as calcium carbonate (CaCO₃)), magnesium carbonate, barium sulfate, zirconium dioxide, polymers (such as nylon) or polymer beads consisting of such polymers, silica (such as fumed silica, silicic acid, or anhydrous silica), silica aluminate, and calcium silicate, which may be surface-treated. Such a reinforcing material can improve physical properties (such as strength) of the body-correcting film, and can also serve as a viscosity modifier. Among the reinforcing materials, a surface-treated silica, such as a silica treated with a surface treatment agent such as hexamethyldisilazane, polydimethylsiloxane, hexadecylsilane, or methacrylsilane, is preferred. Fumed silica is also preferred, and for example, fumed silica surface treated with hexamethyldisilazane or the like can also suitably be used.

In one embodiment, the reinforcing material can have a specific surface area of from 50 to 500 m²/g. The specific surface area of the reinforcing material is preferably from 100 to 350 m² and more preferably from 135 to 250 m²/g. The specific surface area of the reinforcing material can be calculated using the BET method.

In one embodiment, the reinforcing material can have an area circular equivalent particle diameter of from 1 nm to 20 μm. The area circular equivalent particle diameter of the reinforcing material is preferably from 2 nm to 1 μm, and more preferably from 5 nm to 50 nm. Here, the area circular equivalent particle diameter of the reinforcing material can be intended, for example, to be the particle diameter when converted into a circular particle having the same area as projected area of a reinforcing material particle observed with a transmission electron microscope. Such an area circular equivalent particle diameter can be defined as an average value of 10 or more particles.

The blending amount of the reinforcing material with respect to the total amount of the first agent or the second agent can be, for example, 0.0010% by mass or more, 0.010% by mass or more, 0.1% by mass or more, 0.5% by mass or more, 1% by mass or more, 3% by mass or more, or 5% by mass or more, and can be 25% by mass or less, 15% by mass or less, or 10% by mass or less.

From the viewpoint of reinforcing properties and the like of the body-correcting film, the mass ratio of the total amount of the first and second unsaturated organopolysiloxanes, the first and second hydride functionalized polysiloxanes, and the other polymers to the reinforcing material can be from 100:1 to 1:1, and preferably from 50:1 to 2:1, more preferably from 15:1 to 3:1, still more preferably from 10:1 to 4:1, and particularly preferably from 5:1 to 9:1.

At least one selected from a pigment, a dye, and a reinforcing material among the optional ingredients is preferably blended in a first agent. In particular, in cases in which a pigment and a dye are blended into a second agent, when this second agent is applied to the surface to which a first agent is applied, the pigment or the dye may harden during the application, and the pigment or the dye may easily become localized, which may cause color unevenness. From the viewpoint of suppressing color unevenness, it is advantageous for a pigment and a dye to be blended in a first agent. A pigment, a dye, and a reinforcing material may be blended in a second agent to an extent that color unevenness does not occur, and it is advantageous for them not to be contained in the second agent.

### <<Method for Using Application Type Body-Correcting-Film Forming Agent>>

The application type body-correcting-film forming agent of the present disclosure can be used, for example, for cosmetic or medical purposes. Here, the method for using the application type body-correcting-film forming agent of the present disclosure does not encompass a method of operating, treating, or diagnosing a human.

The method for using the application type body-correcting-film forming agent of the present disclosure is not particularly limited and comprises, for example, the following steps. According to the method, it is possible to promote skin permeation of a water-soluble agent. Thus, the method can be referred to as the method for facilitating skin permeation of a water-soluble agent, comprising:
(a) a step of preparing an application type body-correcting-film forming agent comprising: a first agent comprising a cross-linking reactive ingredient that constitutes a body-correcting film; and a second agent comprising a catalyst for cross-linking the cross-linking reactive ingredient;
(b1) a step of applying the first agent to the skin to form a first agent layer, and then applying the second agent to the first agent layer to allow cross-linking, to form a body-correcting film;
(b2) a step of applying the second agent to the skin to form a second agent layer, and then applying the first agent to the second agent layer to allow cross-linking, to form a body-correcting film; or
(b3) a step of mixing the first agent and the second agent to prepare a mixture, and then applying the mixture to the skin to allow cross-linking, to form a body-correcting film, wherein at least one of the first agent and the second agent comprises a water-soluble agent.

From the viewpoint of obtaining a uniform body-correcting film having little unevenness, the method of use is preferably a method in which the first agent is applied to the skin to form a first agent layer, and then the second agent is applied on this first agent layer to allow cross-linking, to form a body-correcting film. Here, with respect to the first agent, the second agent and the water-soluble agent, the materials described above can be used in the same manner.

This method may be performed only once, or may be performed a plurality of times on the formed body-correcting film.

In some embodiments, before the first agent, the second agent, or the mixture containing the first agent and the second agent is applied to the skin, a cosmetic may be applied to the skin; after the first agent is applied to the skin to form the first agent layer, and a cosmetic is applied on the first agent layer, the second agent may be applied to cover the cosmetic; after the second agent is applied to the skin to form the second agent layer, and a cosmetic is applied on the second agent layer, the first agent may be applied to cover the cosmetic; or after the body-correcting film is formed, a cosmetic may be applied to the film.

The cosmetic is not particularly limited. For example, a skin care cosmetic such as a beauty serum, a skin lotion, or a milky lotion; a sunscreen cosmetic (sun block cosmetic); a base cosmetic; or a make-up cosmetic such as a foundation, a gloss, a lipstick, an eye shadow, or a manicure; or a cosmetic having a combination of two or more functions of these cosmetics; can be used.

In some embodiments, a method for using an application type body-correcting-film forming agent of the present disclosure may also be utilized as a beauty method. For example, skin exposed to dryness may lose moisture without realizing it, and the stratum corneum on the surface of the skin may not be able to retain moisture. When the skin lacks moisture, the natural moisturizing factor (NMF) self-produced by the skin cannot be adequately produced. As a result, the barrier function and moisturizing function on the skin surface decrease, making the skin more susceptible to damage, which is thought to cause a loss of moisture and skin roughness.

Meanwhile, by applying a film formed with the application type body-correcting-film forming agent of the present disclosure to the skin, the occlusion effect (the effect that prevents moisture from leaving the skin) of the film can keep the skin well moisturized. As a result, for example, the skin's function of self-producing moisturizing ingredients is improved, and poor turnover in the stratum corneum is also enhanced, making it less likely that problems such as rough skin will occur, thereby increasing the beauty effect. In addition, a film formed with the application type body-correcting-film forming agent of the present disclosure can also improve the skin permeability of a water-soluble agent (e.g., a moisturizer or whitening agent), making it possible to enhance further the skin beauty effects (e.g., moisturizing effect and whitening effect) The term “beauty method” means application of the application type body-correcting-film forming agent of the present disclosure to the skin to form a body-correcting film to beautify conditions of the skin, or a method thereof, which is different from a method of operating, treating, or diagnosing a human.

The method of applying the first agent or the second agent to the skin or the cosmetic-applied layer, or to the first agent layer or the second agent layer, is not particularly limited, and for example, a means of spreading it with a finger or the like, spray application, transfer, or the like can be employed for the application.

When the first agent and/or the second agent show separation into water and oil, it is preferred to shake these agents to force them to be a two-phase system (oil-in-water or water-in-oil) from the viewpoints of cross-linking reactivity between the first agent and the second agent, dispersibility of water droplets containing a water-soluble agent within the film, and skin permeability of such a water-soluble agent, and the like.

### <Application Site>

The application type body-correcting-film forming agent of the present disclosure can be applied on the surface of a skin in any part of a body, namely, on any part of the body surface. For example, the composition can be applied to the skin surface of a head, a face (a lip, an eye, a nose, a cheek, a forehead, or the like), a neck, an ear, a hand, an arm, a leg, a foot, a chest, a belly, a back, buttocks, or the like, as appropriate. Here, the skin also encompasses, for example, a nail, which is formed as a result of hardening due to a change in keratin of the epidermis of a skin.

### <<Kit Containing Application Type Body-Correcting-Film Forming Agent>>

The application type body-correcting-film forming agent of the present disclosure may be provided as a kit containing the above-described first and second agents constituting such a forming agent. In addition to the first agent and the second agent, the kit may also contain an optional member, such as a member to facilitate application of the first agent or the like to a body surface, or various cosmetics described above.

Examples of an optional member include a usage instruction, a brush, a cotton swab, a cutter, scissors, various cosmetics described above, a cleanser for removing the body-correcting film from a body surface, and a mirror. Here, “usage instruction” can encompass, in addition to a general usage instruction attached to the kit in the form of a document, for example, an instruction printed on a packaging container that contains the kit or on a packaging container such as a tube that injects the first agent or the like.

In one embodiment, to prevent contact between the first agent and the second agent, for example, these agents may be contained in separate containers, or may be contained in separate compartments of a container having two or more compartments, in the kit. These agents contained may be configured such that they are applied one at a time, or mixed together before or at the time of use.

### <<Body-Correcting Film>>

### <Thickness>

The thickness of the body-correcting film prepared using the application type body-correcting-film forming agent of the present disclosure described above is not particularly limited, and can be adjusted as appropriate, taking into consideration, for example, breathability, invisibility, compressibility, and closure to a skin. The thickness of the body-correcting film can be, for example, 0.5 μm or more, 1 μm or more, 10 μm or more, 30 μm or more, or 40 μm or more. The upper limit of the thickness is not particularly limited, and can be, for example, 150 μm or less, 100 μm or less, 90 μm or less, 80 μm or less, 70 μm or less, 60 μm or less, or 50 μm or less. Here, the thickness can be defined as an average value calculated by measuring the thickness of any portion of the body-correcting film five times using a High-Accuracy Digimatic Micrometer (MDH-25 MB, manufactured by Mitutoyo Corporation).

### <Performances>

A body-correcting film prepared from the application type body-correcting-film forming agent of the present disclosure can provide excellent results on a variety of performances such as those shown below.

### (Adhesive Strength)

In some embodiments, the obtained body-correcting film can exhibit favorable adhesive strength to a body surface. Such adhesive strength can be evaluated alternatively as the adhesive strength of the body-correcting film applied on a polypropylene substrate. The adhesive strength of the body-correcting film on the polypropylene substrate can achieve 2 N/m or more, 5 N/m or more, 8 N/m or more, 10 N/m or more, or 15 N/m or more. The upper limit of such an adhesive strength is not particularly limited, and, for example, the upper limit can be, from the viewpoint of peelability from a skin or the like, 200 N/m or less, 100 N/m or less, 80 N/m or less, 50 N/m or less, or 30 N/m or less. Here, the adhesive strength can be measured using an Instron device in accordance with the ASTM C794 peel-off adhesion test.

### (Tensile Strength)

In some embodiments, the obtained body-correcting film can exhibit favorable tensile strength. The tensile strength of the body-correcting film can achieve 0.05 MPa or more, 0.10 MPa or more, 0.20 MPa or more, or 0.50 MPa or more. The upper limit of the tensile strength is not particularly limited, and can be, for example, 5.0 MPa or less, 3.0 MPa or less, 2.0 MPa or less, or 1.0 MPa or less. Here, the tensile strength can be measured using an Instron device in accordance with ASTM D5083 elongation tensile test.

### (Elongation at Break)

In some embodiments, the obtained body-correcting film can exhibit favorable elongation at break. The elongation at break of the body-correcting film can achieve 25% or more, 50% or more, 100% or more, 200% or more, or 400% or more. The upper limit of the elongation at break is not particularly limited, and can be, for example, 1,500% or less, 1,200% or less, 1,000% or less, 800% or less, or 600% or less. Here, the elongation at break can be measured using an Instron device in accordance with ASTM D5083 elongation tensile test.

### (Tearing Resistance)

In some embodiments, the obtained body-correcting film can exhibit favorable tearing resistance. Such performance can be evaluated, for example, based on the occurrence or non-occurrence of tearing of the body-correcting film at the time of peeling off from the skin. The occurrence of tearing of the body-correcting film can achieve 15% or less, 10% or less, or 5% or less of the total. The lower limit of the occurrence of tearing is not particularly limited, and can be, for example, 0% or more, or more than 0%.

### (Oxygen Permeability Coefficient)

In some embodiments, the obtained body-correcting film can exhibit a favorable oxygen permeability coefficient. In a body-correcting film having a thickness of 300 μm, the oxygen permeability coefficient of the body-correcting film can achieve 5×10⁻⁹ cm³/(cm²·s) or more, 5×10⁻⁷ cm³/(cm²·s) or more, or 5×10⁻⁵ cm³/(cm²·s) or more. The upper limit of the oxygen permeability coefficient is not particularly limited, and can be, for example, 5 cm³/(cm²·s) or less, 0.5 cm³/(cm²·s) or less, 5×10⁻² cm³/(cm²·s) or less, 5×10⁻³ cm³/(cm²·s) or less, or 5×10⁻⁴ cm³/(cm²·s) or less. Here, the oxygen permeability coefficient can be measured using a Mocon device in accordance with the ASTM F2622 oxygen gas permeability coefficient test for plastic films and sheets.

### (Water Vapor Permeability Coefficient)

In some embodiments, the obtained body-correcting film can exhibit a favorable water vapor permeability coefficient. In a body-correcting film having a thickness of 300 μm, the water vapor permeability coefficient of the body-correcting film can achieve 1×10⁻⁹ cm³/(cm²·s) or more, 1×10⁻⁸ cm³/(cm²·s) or more, or 1×10⁻⁷ cm³/(cm²·s) or more. The upper limit of the water vapor permeability coefficient is not particularly limited, and can be, for example, 1.5×10⁻¹ cm³/(cm²·s) or less, 1.5×10⁻² cm³/(cm²·s) or less, 1×10⁻⁴ cm³/(cm²·s) or less, 1×10⁻⁵ cm³/(cm²·s) or less, or 1×10⁻⁶ cm³/(cm²·s) or less. Here, the water vapor permeability coefficient can be measured using a Mocon device in accordance with the ASTM F1249 water vapor permeability coefficient test for plastic films and sheets.

### EXAMPLES

The present invention will be described in more detail by way of Examples below, but is not limited thereto.

### <<Evaluation Test>>

The permeability test shown below was performed using test samples obtained by the production method described below, and the results are summarized in Tables 1 to 3. Here, “O/W” and “W/O” in the table mean “oil-in-water emulsion composition” and “water-in-oil emulsion composition”, respectively. The “ratio of the intramembrane concentration” means the ratio of the intramembrane concentration in Examples in the tables to the intramembrane concentration in Comparative Examples in the tables.

### <Permeability Test>

The intramembrane concentrations of various water-soluble agents in the epidermis and dermis after 24 hours were measured using the following conditions and methods with a stationary Franz diffusion cell and human skin.

### (Test Condition)

Cell type: Stationary Franz diffusion cell (manufactured by Biocom Systems, Inc.)
Receptor well volume: 3.3 mL
Target skin area: 0.55 cm²
Membrane type: Human skin having a thickness of 300 μm (acquired from New York Firefighter's Skin Bank, Science Care, Allosource, or BioIVT)
Number of skin donors: 1 individual
Details of donors: Gender: male or female; age: from 30 to 70 years old; site: thigh or upper torso
Receptor fluid: Phosphate buffered saline (PBS) containing 0.01% w/w NaN₃ (preservative)

### (Test Method)

The concentrations of water-soluble agents were measured in the receptor chamber of the diffusion cell at different time points. At the end of the diffusion test, the skin was sequentially tape-stripped and divided into epidermal and dermal layers. Concentrates of various water-soluble agents in each of the grouped tape strips and epidermal and dermal tissues were extracted using an extraction solvent and analyzed on an Agilent G6120 HPLC system equipped with an LC-MS detector.

The obtained skin was cryopreserved in a freezer at −20° C. until the morning of the test. Before use, the skin was removed from the freezer and completely thawed at room temperature. Only visually intact areas of the skin were used during testing.

The receptor fluid was prepared at the appropriate pH and degassed by filtering the receptor fluid through a ZapCap (registered trademark) CR 0.2 μm membrane while vacuuming.

The diffusion cell was assembled according to (1) to (10) below. The receptor fluid was maintained at 32° C.±0.5° C. using a stirring-type dry block heater during the test, and the liquid was continuously stirred with a stirrer.
(1) The skin was removed from the freezer and thawed in a biosafety hood for 30 minutes. The skin was completely thawed before opening the package.
(2) The skin was removed from the package and placed on the counter of a biosafety hood with the stratum corneum side up. The skin was patted dry with Kimwipes (registered trademark), then sprayed with fresh PBS and patted dry again. This process was repeated three more times to remove any residue present on the skin.
(3) The receptor well was then filled with degassed receptor fluid. A Teflon (trademark)-coated stirrer bar was placed in each receptor well.
(4) The thawed skin was examined, and only areas with uniform thickness and no visible damage to the surface were used.
(5) The skin was cut into approximately 2 cm square pieces.
(6) The cut skin piece was placed in the center of the donor cell with the stratum corneum side up.
(7) The skin was centered again, and the edges were completely flattened. The donor and receptor wells were then aligned and clamped together with pinch clamps.
(8) Additional receptor fluid was added if necessary. Any air bubbles present were removed by inverting the cell such that air could escape along the sample port.
(9) The diffusion cell was then placed in the stirring-type dry block heater and rehydrated from the receptor fluid for 20 minutes. The block heater was maintained at 32° C.±0.5° C. throughout the test with continuous stirring.
(10) After 20 minutes, the skin surface was examined. In a case in which the skin was moist or showed signs of “sweating”, the stratum corneum was considered damaged and discarded.

Once the cell was assembled and the skin was hydrated for 20 minutes, a tritiated water test was performed to assess the barrier integrity of each skin piece before applying the test sample to the skin. Skin barrier integrity was evaluated according to (a) to (i) below. In addition, regarding skin barrier integrity, detailed information is provided in Tioga Research SOP Lab.011.
(a) A 150 μL aliquot of tritiated water (spiked with 25 μCi water/10 mL water) was added to the donor well of each diffusion cell.
(b) After 5 minutes, the tritiated water from the donor well was removed, and the skin was patted dry using Kimwipes.
(c) After removing the tritiated donor solution, the receptor well was stirred for an additional hour.
(d) After stirring for 1 hour, 300 μL of the aliquot sample was taken from each receptor well. The remaining receptor fluid was discarded and replaced with fresh PBS (the skin barrier integrity test only uses PBS in the receptor fluid).
(e) Each sample aliquot was supplemented with 600 μL of scintillation cocktail (Ultima Gold (trademark) XR).
(f) The tritium content in the receptor well aliquot was then measured using a liquid scintillation counter (LSC).
(g) After the LSC analysis was completed, the results were analyzed. Any diffusion cell exhibiting an abnormally high tritiated water flux was discarded.
(h) The diffusion cells were then ranked according to the tritiated water flux. The diffusion cells were then distributed such that each test sample was assigned to a diffusion cell with a substantially equal average tritiated water flux value.
(i) Once the skin barrier integrity test was terminated, the entire volume of the receptor chamber was replaced with the receptor fluid.

Once the skin barrier integrity test has been terminated and the cells have been properly sorted, it can be said that the test sample is ready to be applied to the stratum corneum of the skin. The donor cell was first removed from each diffusion cell. This step is necessary to allow proper administration of the test sample over the exposed surface area. A plastic washer having an opening of about 0.55 cm² was then placed over the skin such that the opening was aligned with the receptor chamber. A one-time administration regimen was then used in this test. For example, in the case of administering a test sample of an emulsion composition containing a water-soluble agent, 5 μL of the emulsion composition was applied to the skin and spread on the skin surface using a glass rod (care was taken to ensure that the test sample remained within the plastic gasket). In the case of administering a test sample of the application type body-correcting-film forming agent comprising a first agent containing a water-soluble agent and a second agent, 5 μL of the first agent is applied to the skin and spread using a glass rod to form a first agent layer, and then 5 μL of the second agent was applied to the first agent layer. In administering all test samples, the weight of the diffusion cell was measured before and after each administration step to determine the amount of the test sample remaining after spreading.

At 1, 2, 4, 6, 20, and 24 hours, 300 μL sample aliquots were withdrawn from the receptor wells using a graduated Hamilton-type injector syringe. A fresh receptor medium was added to replace the 300 μL sample aliquots. Each sample was then filtered through a 0.2 μm GHP membrane filter plate.

At 24 hours, the skin was patted dry with Kimwipes soaked in PBS/EtOH. A piece of Mepitac (trademark) tape was then applied to the skin, allowed to stand still for 10 minutes, and then removed. This Mepitac (trademark) tape step was repeated to remove the membrane (skin tissue) containing the water-soluble agent completely. After removing the second Mepitac (trademark) tape, the skin was serially tape-stripped. This involved applying a strip of cellophane tape to the skin with light pressure, then removing and retrieving the tape. Each tape strip removed a layer of stratum corneum. Ten tape strips were sampled per cell. The tape strips were grouped into the following sections:
tape strip (TS) 1, tape strip 2, tape strip 3, tape strip 4, tape strip 5, and tape strips 6 to 10.

After tape-stripping the skin, the epidermis of each skin strip was separated from the underlying dermal tissue using tweezers. Epidermal and dermal tissues were collected and placed separately into 4 mL borosilicate glass vials.

After all tape strips and skin pieces were collected, the water-soluble agent was extracted from the tape strips or skin. For the tape strips, this consisted of adding 4 mL of methanol to each vial and stirring the vial at room temperature for 24 hours, after which the sample was collected. For the skin pieces, extraction was performed by adding 2 mL of dimethyl sulfoxide (DMSO) to the vial containing the skin piece and then incubating the vial for 24 hours at 40° C. with gentle stirring. After 24 hours, each sample aliquot was taken and filtered through a 0.20 μm GHP membrane filter plate.

The sample aliquots were analyzed on an Agilent G6120 HPLC system equipped with an LC-MS detector. The samples were refrigerated from 4° C. to 8° C. before analysis, thereby preventing undesired degradation of the water-soluble agent.

### Examples 1 to 5 and Comparative Examples 1 to 3

Each test sample in Examples 1 to 5 and Comparative Examples 1 to 3 was prepared according to the following method.

### Comparative Example 1

An aqueous-phase part was prepared by uniformly mixing: 77.8 parts by mass of ion-exchanged water; 5 parts by mass of 1,3-butylene glycol, 5 parts by mass of dipropylene glycol, and 5 parts by mass of glycerin as moisturizers; 0.2 parts by mass of sodium stearoylmethyltaurate as a surfactant; and 1 part by mass of potassium 4-methoxysalicylate as a water-soluble agent. Then, 3 parts by mass of liquid paraffin, 2 parts by mass of pentaerythrityl tetraethylhexanoate, and 1 part by mass of hydrogenated palm oil were added as oils and uniformly mixed with the aqueous-phase part. Thus, an oil-in-water emulsion composition of Comparative Example 1 was prepared.

### Example 1

### (First Agent)

An oil-phase part was prepared by uniformly mixing 30 parts by mass of divinyl dimethicone at 165,000 cSt as a first unsaturated organopolysiloxane, 6 parts by mass of hydrogen dimethicone at 45 cSt as a first hydride functionalized polysiloxane, 5 parts by mass of silica silylate as a reinforcing material, and 40 parts by mass of a mixture of dimethicone and trisiloxane as an oil. An aqueous-phase part was prepared by uniformly mixing: 13 parts by mass of ion-exchanged water; 2 parts by mass of glycerin, 2 parts by mass of dipropylene glycol, and 1 part by mass of polyethylene glycol as moisturizers; and 1 part by mass of potassium 4-methoxysalicylate as a water-soluble agent. Then, the aqueous-phase part was added and uniformly mixed with the oil-phase part. Thus, a first agent as the water-in-oil emulsion composition was prepared.

### (Second Agent)

An aqueous-phase part was prepared by uniformly mixing 65.5 parts by mass of ion-exchanged water, 20 parts by mass of glycerin as a moisturizer, and 10 parts by mass of ethanol. Next, an oil-phase part was prepared by uniformly mixing 2 parts by mass of bisvinyl dimethicone as an oil, 1 part by mass of PEG-12 dimethicone as a surfactant, and 1.5 parts by mass of a mixture of vinyl dimethicone, divinyl disiloxane, and a platinum catalyst as a catalyst. The oil-phase part was added to the aqueous-phase part, and the resulting mixture was uniformly mixed to prepare a second agent as an oil-in-water emulsion composition.

TABLE 1
(using potassium 4-methoxysalicylate as a water-soluble agent)

| | | | Intramembrane | | Ratio of | |
|---|---|---|---|---|---|---|
| | | Dos- | concentration | | intramembrane | |
| | | age | (μg/cm²) | | concentration | |
| | | form | Epidermis | Dermis | Epidermis | Dermis |
| Comparative | | O/W | 7.6 | 0.5 | — | — |
| Example 1 | | | | | | |
| Example 1 | First agent | W/O | 52.6 | 0.8 | 6.9 | 1.6 |
| | Second | O/W | | | | |
| | agent | | | | | |

### <Results>

The results of Table 1 confirmed that the permeability of a water-soluble agent into the skin can be improved by applying a body-correcting-film forming agent containing a water-soluble agent to the skin to form a body-correcting film rather than by simply applying an oil-in-water emulsion composition comprising a water-soluble agent to the skin.

It was also revealed that potassium 4-methoxysalicylate of the water-soluble agent tends to remain in the epidermis when it permeates the skin.

### Comparative Example 2

An oil-in-water emulsion composition of Comparative Example 2 was prepared in the same manner as in Comparative Example 1, except that the water-soluble agent was changed from 1 part by mass of potassium 4-methoxysalicylate to 1.6 parts by mass of glycylglycine. The increase of the water-soluble agent was adjusted by reducing the amount of ion-exchanged water.

### Example 2

### (First Agent)

A first agent as a water-in-oil emulsion composition was prepared in the same manner as in Example 1, except that the water-soluble agent was changed from 1 part by mass of potassium 4-methoxysalicylate to 1.6 parts by mass of glycylglycine. The increase of the water-soluble agent was adjusted by reducing the amount of ion-exchanged water.

### (Second Agent)

An oil-phase part was prepared by uniformly mixing 5 parts by mass of bisvinyl dimethicone as an oil, 10 parts by mass of cyclopentasiloxane, 1 part by mass of lauryl PEG-9 polydimethylsiloxyethyl dimethicone as a surfactant, and 1.5 parts by mass of a mixture of vinyl dimethicone, divinyl disiloxane, and a platinum catalyst as a catalyst. Next, an aqueous-phase part was prepared by uniformly mixing 62.5 parts by mass of ion-exchanged water, 8 parts by mass of 1,3-butylene glycol, 7 parts by mass of dipropylene glycol, and 5 parts by mass of glycerin as a moisturizer. The aqueous-phase part was added to the oil-phase part, and the resulting mixture was uniformly mixed to prepare a second agent as a water-in-oil emulsion composition.

### Example 3

### (First Agent)

A first agent as a water-in-oil emulsion composition was prepared in the same manner as in Example 1, except that the water-soluble agent was changed from 1 part by mass of potassium 4-methoxysalicylate to 1.6 parts by mass of glycylglycine. The increase of the water-soluble agent was adjusted by reducing the amount of ion-exchanged water.

### (Second Agent)

A second agent as an oil-in-water emulsion composition was prepared in the same manner as in Example 1.

TABLE 2
(using glycylglycine as a water-soluble agent)

| | | | Intramembrane | | Ratio of | |
|---|---|---|---|---|---|---|
| | | Dos- | concentration | | intramembrane | |
| | | age | (μg/cm²) | | concentration | |
| | | form | Epidermis | Dermis | Epidermis | Dermis |
| Comparative | | O/W | 26.6 | 0 | — | — |
| Example 2 | | | | | | |
| Example 2 | First agent | W/O | 29.9 | 1.8 | 1.1 | — |
| | Second | W/O | | | | |
| | agent | | | | | |
| Example 3 | First agent | W/O | 35.2 | 8.7 | 1.3 | — |
| | Second | O/W | | | | |
| | agent | | | | | |

### <Results>

In general, glycylglycine is known as a water-soluble agent that is unlikely to permeate the skin. It was confirmed that the body-correcting-film forming agent of the present disclosure can improve skin permeability even for such an agent.

It was found that in the case of using glycylglycine as a water-soluble agent, skin permeability is further improved when the dosage form of the second agent is an oil-in-water emulsion composition, allowing the second agent to permeate the dermis.

### Comparative Example 3

An oil-in-water emulsion composition of Comparative Example 3 was prepared in the same manner as in Comparative Example 1, except that the water-soluble agent was changed from 1 part by mass of potassium 4-methoxysalicylate to 5 parts by mass of nicotinic acid amide (niacinamide). The increase of the water-soluble agent was adjusted by reducing the amount of ion-exchanged water.

### Example 4

### (First Agent)

A first agent as a water-in-oil emulsion composition was prepared in the same manner as in Example 1, except that the water-soluble agent was changed from 1 part by mass of potassium 4-methoxysalicylate to 5 parts by mass of nicotinic acid amide (niacinamide). The increase of the water-soluble agent was adjusted by reducing the amount of ion-exchanged water.

### (Second Agent)

A second agent as a water-in-oil emulsion composition was prepared in the same manner as in Example 2.

### Example 5

### (First Agent)

A first agent as a water-in-oil emulsion composition was prepared in the same manner as in Example 1, except that the water-soluble agent was changed from 1 part by mass of potassium 4-methoxysalicylate to 5 parts by mass of nicotinic acid amide (niacinamide). The increase of the water-soluble agent was adjusted by reducing the amount of ion-exchanged water.

### (Second Agent)

A second agent as an oil-in-water emulsion composition was prepared in the same manner as in Example 1.

TABLE 3
(using nicotinic acid amide as a water-soluble agent)

| | | | Intramembrane | | Ratio of | |
|---|---|---|---|---|---|---|
| | | Dos- | concentration | | intramembrane | |
| | | age | (μg/cm²) | | concentration | |
| | | form | Epidermis | Dermis | Epidermis | Dermis |
| Comparative | | O/W | 29.1 | 24.5 | — | — |
| Example 3 | | | | | | |
| Example 4 | First agent | W/O | 159.1 | 21.3 | 5.5 | 0.9 |
| | Second | W/O | | | | |
| | agent | | | | | |
| Example 5 | First agent | W/O | 103.3 | 17.2 | 3.5 | 0.7 |
| | Second | O/W | | | | |
| | agent | | | | | |

### <Results>

It was confirmed that even when nicotinic acid amide is used as a water-soluble agent, the body-correcting-film forming agent of the present disclosure can improve skin permeability.

In the case of nicotinic acid amide, it has been found that skin permeability is further improved when the dosage form of the second agent is a water-in-oil emulsion composition.

### REFERENCE SIGNS LIST

10 Moisture
11 Water-soluble agent
12 Solid water-soluble agent
13 Water droplet
14 Body-correcting film

1. An application type body-correcting-film forming agent comprising: a first agent comprising a cross-linking reactive ingredient that constitutes a body-correcting film; and a second agent comprising a catalyst for cross-linking the cross-linking reactive ingredient,
wherein at least one of the first agent and the second agent comprises a water-soluble agent.

2. The forming agent according to claim 1, wherein the water-soluble agent is a water-soluble agent that is in a solid form at room temperature.

3. The forming agent according to claim 2, wherein the water-soluble agent that is in a solid form at room temperature is a crystalline water-soluble agent.

4. The forming agent according to claim 3, wherein the crystalline water-soluble agent is at least one selected from the group consisting of 4-methoxysalicylic acid, tranexamic acid, L-ascorbic acid, 4-methoxysalicylate, tranexamate, L-ascorbate, glycylglycine, nicotinic acid amide, arbutin, L-ascorbic acid glucoside, 1-(2-hydroxyethyl)-2-imidazolidinone, and a pyrimidyl pyrazole compound represented by the following Formula 1 and a salt thereof: 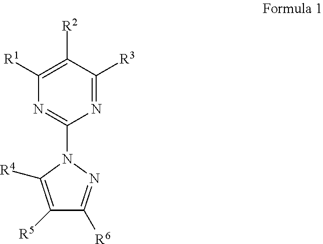
Formula 1
where
R¹, R³, R⁴, and R⁶ are each independently an alkyl group having from 1 to 3 carbon atoms, and
R² and R⁵ are each independently a hydrogen atom or an alkyl group having from 1 to 3 carbon atoms.

5. The forming agent according to claim 1, wherein at least one of the first agent and the second agent comprises the water-soluble agent at 0.1% by mass or more.

6. The forming agent according to claim 1, wherein the first agent comprises at least one selected from the group consisting of a first unsaturated organopolysiloxane and a first hydride functionalized polysiloxane,
wherein when the first agent only comprises the first unsaturated organopolysiloxane out of the first unsaturated organopolysiloxane and the first hydride functionalized polysiloxane, the second agent comprises the second hydride functionalized polysiloxane, and
wherein when the first agent only comprises the first hydride functionalized polysiloxane out of the first unsaturated organopolysiloxane and the first hydride functionalized polysiloxane, the second agent comprises the second unsaturated organopolysiloxane.

7. The forming agent according to claim 6, wherein the first unsaturated organopolysiloxane and the second unsaturated organopolysiloxane are at least one selected from the group consisting of organopolysiloxanes containing a vinyl group, vinyl-terminated organopolysiloxanes, and organopolysiloxanes containing a vinylated branched chain.

8. The forming agent according to claim 7, wherein the first unsaturated organopolysiloxane and the second unsaturated organopolysiloxane are at least one selected from the group consisting of vinyl-terminated polydimethylsiloxanes, vinyl-terminated diphenylsiloxane-dimethylsiloxane copolymers, vinyl-terminated polyphenylmethylsiloxanes, vinylphenylmethyl-terminated vinylphenylsiloxane-phenylmethylsiloxane copolymers, vinyl-terminated trifluoropropylmethylsiloxane-dimethylsiloxane copolymers, vinyl-terminated diethylsiloxane-dimethylsiloxane copolymers, vinylmethylsiloxane-dimethylsiloxane copolymers, trimethylsiloxy-terminated vinylmethylsiloxane-dimethylsiloxane copolymers, silanol-terminated vinylmethylsiloxane-dimethylsiloxane copolymers, vinylmethylsiloxane homopolymers, vinyl T-structure polymers, vinyl Q-structure polymers, monovinyl-terminated polydimethylsiloxanes, vinylmethylsiloxane terpolymers, and vinylmethoxysilane homopolymers.

9. The forming agent according to claim 6, wherein the first hydride functionalized polysiloxane and the second hydride functionalized polysiloxane are non-terminally and/or terminally hydrogenated organopolysiloxanes.

10. The forming agent according to claim 9, wherein the first hydride functionalized polysiloxane and the second hydride functionalized polysiloxane are at least one selected from the group consisting of hydride-terminated polydimethylsiloxanes, hydride-terminated polyphenyl-(dimethylhydrosiloxy)siloxanes, hydride-terminated methylhydrosiloxane-phenylmethylsiloxane copolymers, trimethylsiloxy-terminated methylhydrosiloxane-dimethylsiloxane copolymers, polymethylhydrosiloxanes, trimethylsiloxy-terminated polyethylhydrosiloxanes, triethylsiloxanes, methylhydrosiloxane-phenyloctylmethylsiloxane copolymers, and methylhydrosiloxane-phenyloctylmethylsiloxane terpolymers.

11. The forming agent according to claim 1, wherein the catalyst is at least one selected from the group consisting of a platinum carbonyl cyclovinylmethylsiloxane complex, a platinum divinyltetramethyldisiloxane complex, a platinum cyclovinylmethylsiloxane complex, and a platinum octanaldehyde/octanol complex.

12. The forming agent according to claim 1, wherein at least one of the first agent and the second agent comprises at least one selected from the group consisting of a fiber, a pigment, a dye, a thickener, a UV absorber, and a reinforcing material.

13. The forming agent according to claim 12, wherein the first agent comprises the reinforcing material at 0.001% by mass or more.

14. The forming agent according to claim 1, wherein at least one of the first agent and the second agent is an emulsion composition.

15. The forming agent according to claim 1, which is used for skin permeation of the water-soluble agent.

16. A kit comprising the first agent and the second agent in the forming agent according to claim 1, wherein the first agent and the second agent are contained in separate containers, or contained in separate compartments of a container having two or more compartments.

17. A method for facilitating skin permeation of a water-soluble agent, comprising:
(a) preparing an application type body-correcting-film forming agent comprising: a first agent comprising a cross-linking reactive ingredient that constitutes a body-correcting film; and a second agent comprising a catalyst for cross-linking the cross-linking reactive ingredient;
(b1) applying the first agent to the skin to form a first agent layer, and then applying the second agent to the first agent layer to allow cross-linking, to form a body-correcting film;
(b2) applying the second agent to the skin to form a second agent layer, and then applying the first agent to the second agent layer to allow cross-linking, to form a body-correcting film; or
(b3) mixing the first agent and the second agent to prepare a mixture, and then applying the mixture to the skin to allow cross-linking, to form a body-correcting film,
wherein at least one of the first agent and the second agent comprises a water-soluble agent.
